(19) United States
(12) Patent Application Publication
(10) Pub. No.: US 2023/0166129 A1
(43) Pub. Date: Jun. 1, 2023

(54) HEAD-WEARABLE DEVICES FOR POSITIONING ULTRASOUND TRANSDUCERS FOR BRAIN STIMULATION

(71) Applicant: Attune Neurosciences, Inc., Menlo Park, CA (US)

(72) Inventors: Keith R. Murphy, San Francisco, CA (US); Cameron Good, Joppa, MD (US); Tommaso Di Ianni, San Francisco, CA (US); Rajiv Mahadevan, Menlo Park, CA (US); Pablo Villoslada, Palo Alto, CA (US); Michael W. Metz, Plymouth, MN (US); Laura Ott, Providence, RI (US); Corey Brown, Providence, RI (US); Julia Hines, Providence, RI (US); Ayan Bhandari, Redwood City, CA (US)

(73) Assignee: Attune Neurosciences, Inc., Menlo Park, CA (US)

(21) Appl. No.: 18/071,472

(22) Filed: Nov. 29, 2022

Related U.S. Application Data
(60) Provisional application No. 63/283,958, filed on Nov. 29, 2021.

(51) Int. Cl. A61N 7/00

(52) U.S. Cl. CPC A61N 7/00; A61N 2007/0026; A61N 2007/0034

(57) ABSTRACT

A head-wearable device is configured to hold at least one ultrasound transducer such that it is properly positioned for brain stimulation. Such a head-wearable device can help to ensure that the transducer makes sufficient contact with the user's head to allow for efficient ultrasound coupling. Each device may also ensure that the ultrasound transducer maintains the same orientation and position relative to the head across successive donning and doffing of the device.

### CROSS REFERENCE TO RELATED APPLICATION

This application claims priority to U.S. Provisional Patent Application No. 63/283,958, entitled “Systems and Methods for Reliable Spatial Orientation and Positioning of Ultrasound Arrays for Use in Ultrasonic Brain Stimulation,” and filed on Nov. 29, 2021, which is incorporated herein by reference.

### RELATED ART

Multiple ultrasound waves can constructively interfere, or focus, at a distance from their sources using the same principles that allow light to be focused through a lens. To accomplish this, an array of individual ultrasound elements, or sources, can focus ultrasound waves into a restricted region by assigning different time or phase delays to the waves passed onto each element. These delays can mimic the effect of physical curvature normally used to achieve focusing. The pressure created at the resulting focus can be sufficient to engage mechanosensitive channels which regulate neuronal membrane potential to either inhibit or excite neurons. By targeting the focus onto specific brain regions associated with psychiatric disorders, therapeutic effects can be achieved.

In practice, conventional therapeutic ultrasound devices with anatomically precise targeting often require real-time MRI data acquisition and acoustic simulation to identify the neural target region and properly assign time delays to each ultrasound element to enable focusing on that region. Calculating these delays can be achieved by collecting the coordinates of each ultrasound element and the target region within the MRI space, estimating acoustic properties of that space, and then assessing wave travel time from each element to the target region through time reversal, ray tracing, or similar methods. While these methods have been effective in tissue ablation or in-clinic neuromodulation, they have not typically allowed for out-of-clinic ultrasound use since the device cannot be reliably registered to the user's skull without active MRI. In theory, the ultrasound targeting achieved under MRI guidance should be applicable across uses given the stability of the human brain and skull. However, these clinical devices offer no means of returning ultrasound arrays to the same position and orientation at the time of the subsequent scans. Thus, creation of a device which allows its users to reliably return the position of the transducers would allow focused ultrasound therapy in the absence of an operator or clinician, allowing for use for nightly at home therapeutic use during normal waking activity or sleep.

### BRIEF DESCRIPTION OF THE DRAWINGS

The disclosure can be better understood with reference to the following drawings. The elements of the drawings are not necessarily to scale relative to each other, emphasis instead being placed upon clearly illustrating the principles of the disclosure. Furthermore, like reference numerals designate corresponding parts throughout the several views.

FIG. 1 depicts an ultrasound transducer of a head-wearable device making poor contact with a head of a user.

FIG. 2 depicts the ultrasound transducer of FIG. 1 after the ultrasound transducer has been rotated to make good contact with the head of the user.

FIG. 3A depicts a user wearing a head-wearable device having an exemplary embodiment of a registration tool for positioning the head-wearable device on a head of the user.

FIG. 3B depicts the registration tool of the head-wearable device depicted by FIG. 3A.

FIG. 4 depicts a user wearing a head-wearable device having another embodiment of a registration tool for positioning the head-wearable device on a head of the user.

FIG. 5A depicts a user wearing a head-wearable device having a registration tool for positioning a transducer to a predefined position and orientation relative to a head of the user.

FIG. 5B depicts the registration tool of FIG. 5A.

FIG. 6A is top view illustrating a transducer having a recess for receiving a base plate of the registration tool depicted by FIG. 5B.

FIG. 6B is a side view illustrating the transducer of FIG. 6A.

FIG. 7 is a block diagram illustrating an exemplary embodiment of a system having an ear hook assembly for positioning a transducer of a head-wearable device.

FIG. 8A depicts an exemplary embodiment of an ear hook assembly, such as is depicted by FIG. 7, while a spring of the ear hook assembly is stretched prior to positioning the ear hook assembly on an ear of a user.

FIG. 8B depicts the ear hook assembly of FIG. 8A after it is positioned on the ear of the user.

FIG. 9 is a block diagram illustrating a system for measuring and indicating the quality of contact between a transducer of a head-wearable device and the skin of a user.

FIG. 10A depicts a transducer having poor contact with the skin of a user.

FIG. 10B depicts the transducer of FIG. 10A when it flush against the skin of the user.

FIG. 11 depicts an exemplary embodiment of a head-wearable device having a strap for pressing a transducer against a head of a user.

FIG. 12 depicts a user wearing a head-wearable device having a registration tool for positioning a transducer to a predefined position and orientation relative to a head of the user.

FIG. 13 depicts a user wearing a head-wearable device having a registration tool for positioning a transducer to a predefined position and orientation relative to a head of the user.

FIG. 14 depicts a rotating ball joint of the registration tool depicted by FIG. 13.

FIG. 15 is a block diagram illustrating an exemplary system for transducer displacement estimation.

### DETAILED DESCRIPTION

The present disclosure generally pertains to head-wearable devices for holding multi-element ultrasound transducer arrays. Such devices can help to ensure that the arrays make sufficient contact with the user's head (e.g., skull) to allow for efficient ultrasound coupling. Each device may also ensure that the ultrasound transducers maintain the same orientation and position relative to the head across successive donning and doffing of the device.

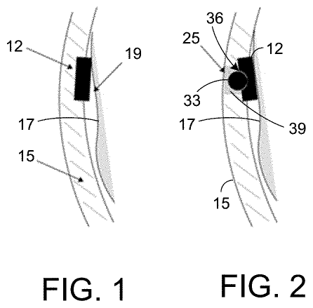

In principle, increasing the rigidity of a wearable device reduces its rotational freedom across uneven or curved surfaces. Thus, the position of an ultrasound transducer in a head mounted wearable would likely become more reliable with the use of rigid structural elements. However, these rigid structures increase the likelihood of floating sections when contours are sharp, as is often true of the temporal window of the skull. For example, FIG. 1 shows a rigid band 15 of a head-wearable device attached to a transducer 12 that is positioned against the head 17 of a user. Due to a curvature of the user's head 17, a portion of the transducer is “floating” such that an air gap 19 exists between such floating portion and the user's head 17.

If the floating section is present at the transducer face, numerous issues arise. For example, if the gap 19 does not contain a viscous ultrasound coupling agent, the ultrasound waves can be almost entirely reflected. This occurs due to the large difference in acoustic impedance between air (Z˜0.0004×10⁶) and skin (Z˜1.53×10⁶). The relationship between this difference and the fraction of ultrasound reflected is given by the equation r=((Z1−Z2)/(Z1+Z2))². High levels of reflection can cause reduced or elimination of treatment efficacy and may also damage the ultrasound transducer. While these reflections can be mitigated with use of a coupling agent such as ultrasound gel, dry ultrasound coupling requires flush contact, even with more flexible material interfaces such as silicone or hydrogels. Assuming coupling, the problem is further complicated by refraction which is proportional to the angle of incidence between two mediums of different acoustic properties.

In some embodiments, a transducer 12 is coupled to a band 15 of a head-wearable device via a ball joint 25, as shown by FIG. 2. The ball joint 25 permits the transducer 12 to rotate relative to the band 15 of the head-wearable device, thereby allowing the surface of the transducer 12 to fit flush against the surface of the user's head 17, as shown by FIG. 2. Thus, the presence of the ball joint 25 between the transducer 12 and the band 15, as shown by FIG. 2, helps to mitigate the free-floating surface issue by rolling the transducer 12 relative to the band 15 to a state of least resistance or strain. This will naturally move the transducer 12 to a substantially flat position against the surface of the user's head 17. Thus, efficient transmission of ultrasound waves from the transducer 12 into the user's brain occurs, thereby improving therapeutic outcomes.

As known, in the art, a ball joint 25 comprises a bearing stud 33, such as a spherical ball bearing, that is situated in a socket 36 enclosed by a casing 39. As shown by FIG. 2, the ball joint 25 couples the transducer to a band or other component of head wearable device, and the ball joint permits the transducer to rotate with 360° of movement. When the transducer 12 is pressed against the user's head by the band 15 of the head wearable device, the ball joint 25 allows the transducer 12 to rotate such that the surface of the transducer 12 facing the user's head is pressed flush against the user's skin across the transducer's surface, as shown by FIG. 2. In other embodiments, other types of interfaces may be used to permit rotation of the transducer 12 in a similar manner so that the transducer's surface is positioned substantially flush against the user's skin.

Returning a transducer 12 to a given transducer placement from one wear to the next requires rotational accuracy. However, to accommodate user functionality, particularly for persons with longer or thicker hair, the head-wearable device can contain straps which can be loosened during the donning of the device. Once the device is on, the straps can be tightened to a predefined measure. While these measures may be predetermined to limit motion of the device, soft structures in the head-wearable device may allow for yaw and pitch rotation under the same amount of adjustment. This would allow the transducer 12 undesirably to change position or orientation across uses which would lead to ultrasound focusing inaccuracies.

In some embodiments of the present disclosure, as will be described in more detail below, a registration tool, such as a “nose fit” tool, attaches to one or more transducers 12 in a spatially-fixed manner so that the transducer can be oriented in a consistent manner from one wear to the next. In order for the nose fit tool to contact body part (e.g., the user's nose bridge) at a given point, the position and the orientation of one or more transducers are constrained to a small degree of freedom. Additional points of contact along the nose can further limit rotational freedom. For instance, a single point of contact at the center (or other point) of the nose can still be maintained while the device changes pitch. In this case, the top banding rising up with hair volume may go unnoticed by the user if the tool contacts the nose bridge at the correct point. However, maintaining contact at two points along the anterior posterior axis requires an exact pitch.

In practice, the user may assess when the nose fit tool is making correct contact and adjust accordingly. The nose fit tool may be present as a fitting tool during the donning procedure and can be removed once the device is snugly fit or straps are tightened to the appropriate levels. It may also be used prior to a locking procedure where the device's position may become immobile on the user's head. The nose fit tool may also stay integrated to the wearable device, either maintaining contact with the nose, or swinging, hinging, or otherwise moving away from the user's face during device operation. In another embodiment, the nose fit tool could be magnetically mounted to the transducer outer surface. The tool position and orientation may then be adjusted using several rotation and motion points at the base of the fit tool.

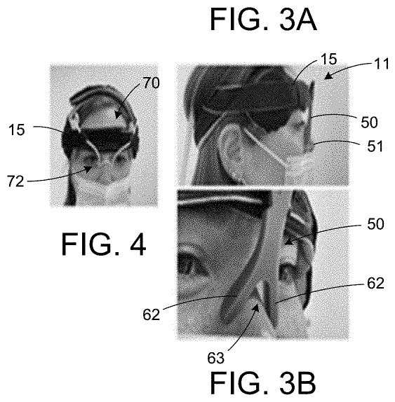

FIGS. 3A and 3B depict an exemplary embodiment of a registration tool 50, referred to hereafter as a “nose fit tool,” that can be used to position a head-wearable device 11 and, thus, components (e.g., transducers) of the head-wearable device 11 to a predefined positon and orientation relative to a head of a user. The nose fit tool 50 has one end coupled to a band 15 of a head-wearable device 11 and another end that is configured to fit on or over a bridge of the user's nose 51. The end that fits on or over the user's nose bridge is forked such that it has a pair of fingers 62 forming a notch 63. The notch 63 may have a “V” or “U” shape where the user's nose 51 may be positioned in the notch 63 between the fingers 62. In use, the fingers 62 contact and press against the user's nose 51 such that the tool 50 holds the band 15 at a predefined distance from the user's nose 51 and at a predefined orientation relative to the user's nose 51. FIG. 4 also shows a nose fit tool 70 in accordance with another embodiment where the tool 70 couples to the band 15 at multiple points. The tool 70 has a curved portion 72 that forms a notch for receiving a bridge of the user's nose 51, as described above for the notch 63 of the tool 50. As shown by FIG. 4, the portion of tool 70 that fits over the user's nose 51 forms a “W,” with a notch in the middle of the “W” for receiving the bridge of the user's nose 51.

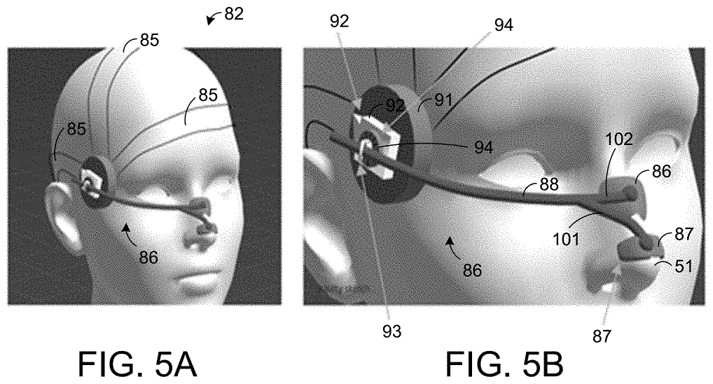

FIGS. 5A and 5B show a head-wearable device 82 having a nose fit tool 86 in accordance with another embodiment. As shown by FIGS. 5A and 5B, the head-wearable device 82 has a plurality of bands 85 that fit around the head of the user to hold the device 82 in place. The nose fit tool 86 comprises a pair of nose pads 86, 87 that are curved for fitting around and making flush contact with the bridge of the user's nose 51 at two different points. The pads 86, 87 are coupled to a transducer 91 by a fiducial contact arm 88 that extends from an arm holder 93 to each of the pads 86, 87. In this regard, the arm 88 is forked as shown to provide a pair of ends 101, 102, each of which is coupled to a respective pad 86, 87. The arm holder 93, which in the embodiment shown by FIG. 5B forms a cylindrical loop for receiving the arm 88, is mounted on a rotating joint 94. In FIG. 5B, the rotating joint 94 is implemented as a disc, but other configurations of the rotating joint 94 are possible in other embodiments. The rotating joint 94 is coupled to a base plate 92 that is attached to the transducer 91. In some embodiments, the base plate 92 is composed of magnetic material that generates a magnetic force for holding the plate 92 against the transducer 91. As an example, the transducer 91 may have a metallic plate or other metallic component such that a magnetic force between the magnetic base plate 92 and the metallic component is sufficient for holding the base plate 92 on the transducer 91. In other embodiments, it is unnecessary for magnetic force to be used to couple the arm holder 73 to the transducer 91, and the base plate 92 may be composed of non-magnetic material.

The rotating joint 94 may be rotated as desired to enable the pads 86, 87 to be appropriately positioned on the user's nose as shown. Once this has been achieved, the rotating joint 94 may be locked such that further rotation of the joint 94 relative to the base plate 92 is prevented. As an example, the rotating joint 94 may be taped or glued to the base plate 92 in order to lock the joint 94 in place. Coupling devices, such as screws, may also be used to lock the join 94 in place.

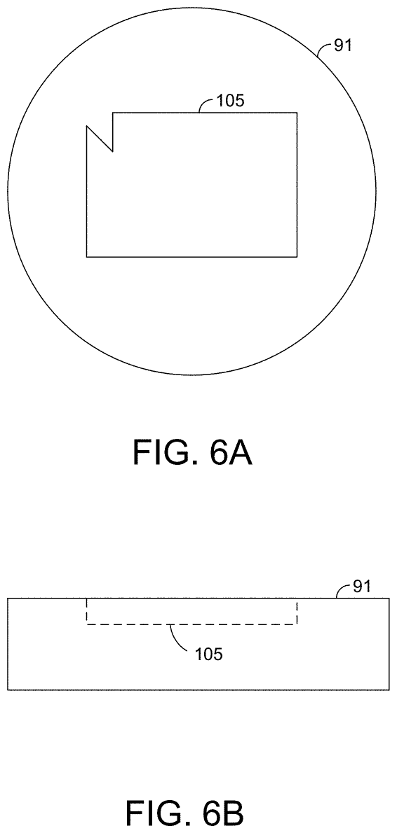

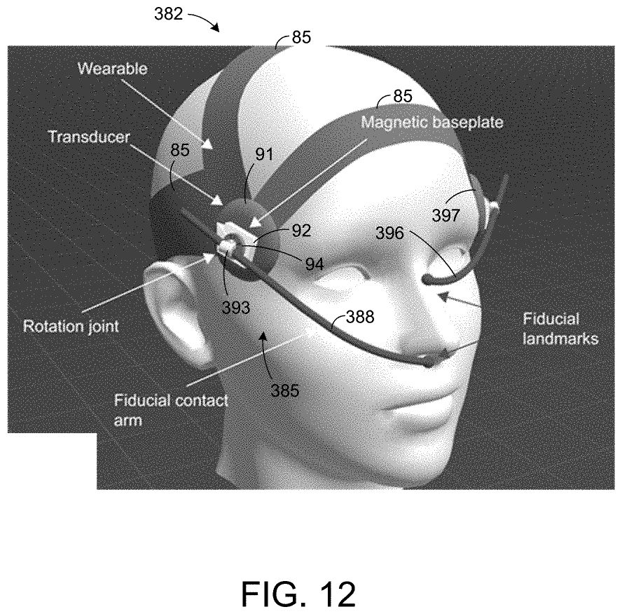

The transducer 91 may have a recess 105 (FIG. 6A) for receiving the base plate 92 where the boundary of the recess 105 has the same shape as the outer perimeter of the base plate 92 such that the base plate 92 snugly fits in the recess 105. Further, the shape (e.g., a perimeter) of the base plate 92 and, thus, the recess 105 that has the same shape as the base plate 92 may be irregular or otherwise asymmetrical such that the base plate 92 fits into the recess 105 in only one orientation relative to the transducer 91. Thus, the base plate 92 may be removed from the transducer 91 after fitting, if desired. Later, in order to properly align or otherwise position the transducer 91, such as when the user dons the head-wearable device at a later time, the base plate 92 may be attached to the transducer 91 via magnetic force from the magnetic base plate 92 by placing the base plate 92 into the recess 105 of the transducer 91. In this regard, the user orients the base plate 92 such that it fits into the recess 105 similar to a piece of a jigsaw puzzle, noting that an irregular or asymmetrical shape of the base plate 32 and recess 105 helps to ensure that the base plate 92 and thus the arm 88 that is coupled to the base plate 92 are in the same orientation relative to the transducer 91 as during fitting. Other registration tools similar to the one shown by FIGS. 5A and 5B will be described in more detail below with reference to FIG. 12.

Note that the same techniques described above may be used to properly orient the base plate 92 without using magnetic force. As an example, rather than using magnetic force, the base plate 92 may be attached to the transducer 91 with screws or other coupling devices.

Transducers in a head-wearable device may be subject to anterior/posterior (AP) motion relative to the temporal window or rotation around the lateral/ipsilateral (LI) axis. To limit this motion, the ear can serve as an anchor point for a head-wearable device and attached transducers by fixing AP-positional and LI-axial motion. In some embodiments, an ear hook assembly is used that is adjustable in the AP dimension via spring loading (e.g., using a spring-bolt mechanism). The ear hook assembly can be adjusted in the AP direction for each user's anterior posterior ear position. To allow for releasing the hook or easy placement and a snug fit during wear, the ear hook assembly holds tension using a spring which can be stretched during the donning procedure and relaxed when in proper place. If the device is not in the proper AP position, the user will feel tension on the spring as a force at the back of the ear. This signals the user that the transducers are too anterior and that the head wearable device should be adjusted. In addition to the user feeling spring tension, a force or tension sensor may be integrated to measure the force exerted by the spring to identify the AP fit programmatically.

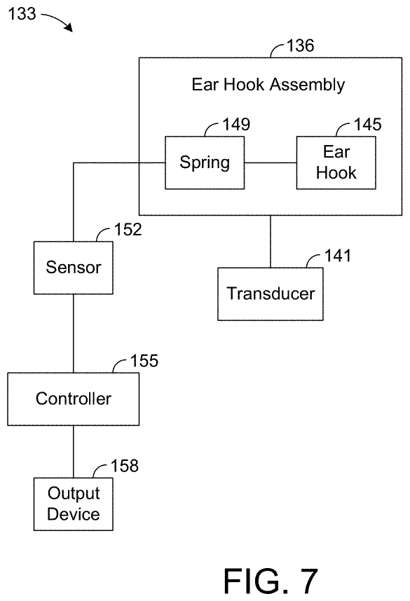

As an example, FIG. 7 depicts an exemplary system 133 that may be integrated with a head-wearable device for controlling a position of a transducer 141. The system 133 has an ear hook assembly 136, which has a curved ear hook 145 that attaches to an ear of a user, as described above. The ear hook 145 is connected to a spring 149 that applies a force to the ear hook 145 when it is displaced, as described above. The spring 149 is coupled to a sensor 152 that measures a force applied by the spring 149. The sensor 152 is coupled to a controller 155 that is configured to receive sensor data from the sensor 152 indicating the measured force, and use this measurement to provide feedback to a user indicating whether the ear hook assembly 136 and, thus, transducer 141 are properly positioned based on the measured force. In this regard, the controller 155 may be coupled to an output device 158, such as a speaker, light source (e.g., one or more LEDs), or display, that provides audible or visible information indicating whether a proper positioning is achieved. As an example, a certain feedback, such as audible or visual alert, may be generated when the measured tension in the spring is within a predefined range indicating that the transducer 141 is at a proper distance from the user's ear. By properly hooking the assembly 136 to the ear, the attached transducer 141 should be oriented correctly around the LI-axis.

Note that the controller 155 may be implemented in hardware or any combination of hardware and software. As an example, the controller 155 may comprise at least one processor executing software for performing functions ascribed to the controller 155 herein. In other embodiments, the controller 155 may be implemented via a field programmable gate array (FPGA) or application-specific integrated circuit (ASIC). Yet other configurations of the controller 155 are possible in other embodiments.

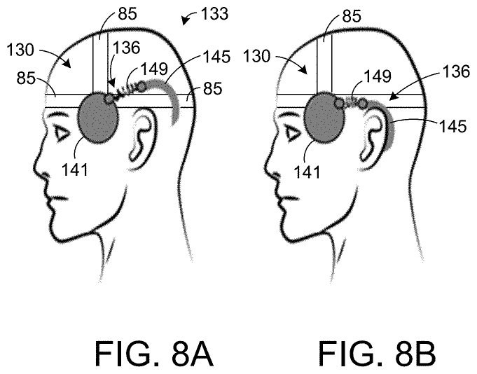

FIGS. 8A and 8B depict an exemplary embodiment of a head-wearable device 130 having an ear hook assembly 136 that may be used to properly position a transducer 141, as described above. As in other embodiments, the head-wearable device 130 may have a plurality of bands 85 for holding the device 130 in place. Further, the ear hook assembly 136 comprises a curved ear hook 145 that is coupled to a transducer 141 by a spring 149. The transducer 141 may be coupled to a band 85 of the head wearable device 130. The ear hook 145 is shaped to fit around the user's ear as shown.

When the head-wearable device 130 is being donned, the user may stretch the assembly 136 by pulling the ear hook 145 away from the transducer 141 to provide more space between the ear hook 145 and the transducer 141 for accommodating the user's ear, as shown by FIG. 8A. Once the ear hook 145 is positioned behind the user's ear and the transducer 141 is positioned approximately at its desired position, the user may release the assembly 136 such that the force of the spring 149 pulls the transducer 141 and the ear hook 145 together, as shown by FIG. 8B. Once the ear hook 145 is in contact with the user's ear, movement of the ear hook 145 by the spring 149 relative to the user's head is prevented by the user's ear such that the force of the spring 149 pulls the transducer 141 to the appropriate location relative to the user's head. As noted above, a sensor 152 (FIG. 7) that measures the force (e.g., tension) applied by the spring 149 may be used to confirm that the tension in the spring 149 has fallen below a threshold thereby indicating that the transducer 141 is at the proper location (i.e., there is no unexpected external force restricting the movement of the transducer 141 so that the transducer 141 can reach the desired distance from the ear hook 145 that is positioned behind the user's ear).

A head-wearable device should induce contact of the ultrasound transducer face, coupling pad on the transducer face, gel between the ultrasound transducer and skin, or some combination of these elements, to the user's skin to a certain extent. However, the presence of hair under the transducer or air gaps resulting from skin folds or inhomogeneities is always possible. It is unlikely that the user would know the extent of these coupling gaps which could impair the efficacy of treatment throughout a session. In some embodiments, a system may be integrated with a head-wearable device for providing feedback quantifying or otherwise indicating the extent of transducer/skin contact which could alert the user of insufficient contact. The system measures some electrical property of a circuit containing both the coupling medium and the patient skin. This coupling medium could be an ultrasound coupling pad, ultrasound gel, the ultrasound transducer faceplate, or the ultrasound transducer itself. In one embodiment, an electrode is placed in contact with the coupling medium and an additional electrode placed somewhere on the patient's skin. Capacitance or resistance of this circuit can then be calibrated to no-contact when the user is instructed to lift the transducer gently off of their skin, and during full contact, when the transducer is carefully placed to avoid any air gaps. During wear, the system will monitor the calibrated property and could alert the user through a tone when the coupling is poor. The user may be alerted through other sensory modalities such as a flashing light or vibration. The detection may also engage an automated system for mechanically adjusting the angle of the transducer to aid in air gap removal.

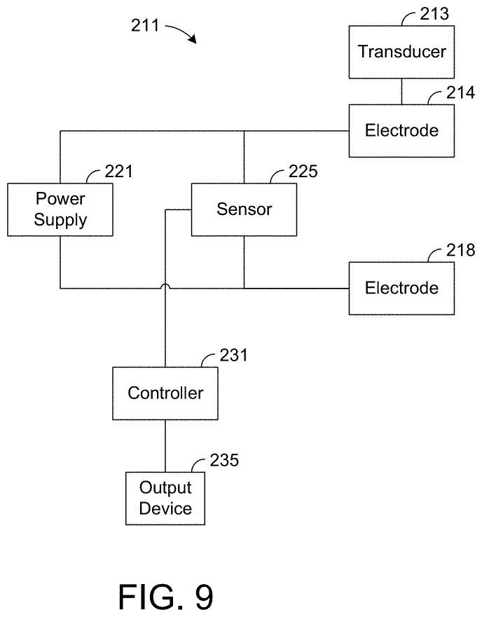

As an example, FIG. 9 shows an exemplary system 211 that may be used to measure and indicate the quality of contact between a transducer 213 of a head-wearable device (not shown in FIG. 9) and the skin of a user. In the embodiment depicted by FIG. 9, an electrode 214 is coupled to the transducer 213, and another electrode 218 is positioned to contact the user's skin. As an example, the electrode 218 may be coupled to a band 85 or other component of a head wearable device (not shown in FIG. 9) that passes over the user's skin such that the electrode 218 contacts the skin (e.g., forehead or other part of the user's head) of the user. The electrodes 214, 218 may be coupled to a power supply 221 (e.g., a battery). In some embodiments, the electrode 214 is a conductive layer or pad of the transducer 213 that makes contact with the user's skin when the transducer 213 is positioned against the user's head.

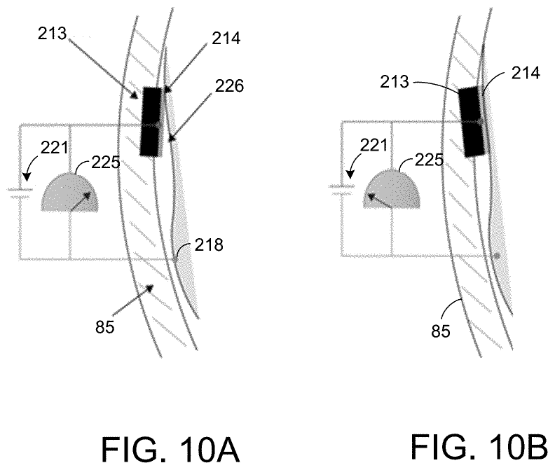

A sensor 225 may be configured to measure an electrical parameter that is affected by the position of the transducer 221 relative to the user's head. As an example, the sensor 221 may measure the current that flows through the electrode 214, user's skin, and the electrode 218. In other embodiments, the sensor 221 may measure capacitance or resistance between the electrodes 214, 218. Flush, firm contact of the transducer 213 and, hence, electrode 214 against the user's skin, as shown by FIG. 10B, should reduce the resistance between the electrodes 214, 218. However, if the transducer 213 is positioned poorly such that firm contact does not occur, as shown by FIG. 10A, which shows a presence of an air gap 226 between the transducer 213 and the user's skin, then the resistance between the electrodes 214, 218 should be greater, there affecting the parameter (e.g., resistance or capacitance) measured by the sensor 225. The sensor measurements may be provided as an input to a controller 231 that determines whether the transducer 213 is properly oriented relative to the user's head based on such input and then provides feedback indicating whether the transducer 213 is properly oriented. As an example, if the measured parameter is within a certain range, the controller 231 may determine that the transducer 213 is properly oriented and provide an output (e.g., an audible of visual alert) indicative of such. Otherwise, the controller 231 may provide an output indicating that the transducer 213 is not properly oriented. In this regard, the controller 231 may be coupled to an output device 235, such as a speaker, light source (e.g., one or more LEDs), or display, that provides audible or visible information indicating whether and/or quantifying an extent to which a proper positioning of the transducer 213 is achieved.

Note that the controller 231 may be implemented in hardware or any combination of hardware and software. As an example, the controller 231 may comprise at least one processor executing software for performing functions ascribed to the controller 231 herein. In other embodiments, the controller 231 may be implemented via a field programmable gate array (FPGA) or application-specific integrated circuit (ASIC). Yet other configurations of the controller 231 are possible in other embodiments.

To help avoid air gaps between a transducer and the user's skin/skull surface, compression force can be applied to the transducer. Questionnaire examinations have shown that subjective comfort rating of clamping headphones is relatively unaffected up to 6 N of compression force, at which point the wearers begin to rate headphones as “a little tight.” Fortunately, 6 N far exceeds the minimum force required to perform ultrasound imaging (0.049 N), with higher levels of force providing only negligible improvements; 0.049 N were used to achieve a 98% liver identification success rate compared to 29.15 N for 100%. To achieve such a compression force, a band may pass the transducer as it moves from the back to the front of the head. By layering this band on top of the transducer, and strapping force applied to the front of the head can serve to secure the head-wearable device while applying compression force to the transducer.

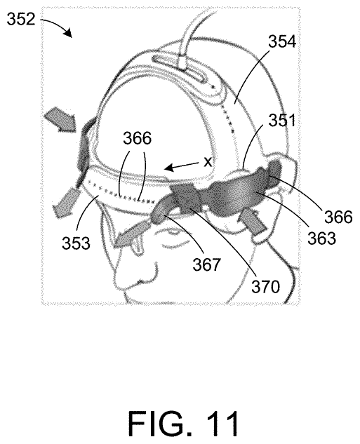

FIG. 11 depicts an exemplary embodiment of a head-wearable device 352 having a strap 363 for applying compression to a transducer 351. The device has a first band 354 that passes over the top of a user's head as shown and a second band 353 that passes over the user's forehead as shown. Both of the bands 352, 353 may be adjustable. The strap 363 may be elastic to permit it to stretch, but it is unnecessary for the strap 363 to be elastic in other embodiments. As shown by FIG. 11, the strap 363 has one end 366 that is attached (e.g., sewn) to the band 353. The opposite end 367 of the strap 363 may be pulled to stretch the strap 363 over the transducer 351 in order to apply a force for pressing the transducer 351 against the user's head. In general, pulling the end 367 further away from the end 66 in the x-direction such that the strap 363 is further stretched causes the strap 363 to apply a greater force to the transducer 351. Once the end 367 is pulled by the desired amount to generate the desired amount of force exerted on the transducer 351, the end 367 may be locked in place by a locking mechanism so that the desired amount of force is continuously applied to the transducer 351 until the locking mechanism is intentionally unlocked by a user.

Various types of locking mechanisms may be used to hold the end 367 in place. In some embodiments, Velcro is used as a locking mechanism. As an example, the band 353 and the end 367 may be composed of a Velcro material (i.e., material with hook-and-loop fasteners) such that the end 367 may be locked in place by simply pressing the Velcro material of the end 367 against the Velcro material of the band 353. As shown by FIG. 11, the end 367 may pass through a loop 370 to help guide the strap end 367 as it is being pulled and to help hold the end 367 in place once it is pressed against the band 353. In other embodiments, other techniques and devices for holding the end 367 in place as may be desired are possible. As an example, the band 353 may have one or more holes 366 through which a protrusion (e.g., a peg) (not shown) from the end 367 may pass in order to secure the end 367 to the band 353. In other embodiments, yet other techniques to secure the strap 363 are possible.

To limit the rotational and positional freedom of an ultrasound transducer relative to the user's head, a single or multiple points of registration with the face and its constituent landmarks can be used. FIG. 12 depicts an embodiment of a head-wearable device 382 having a registration tool 385 that can be used to ensure an ultrasound transducer 91 is positioned at a predefined position and orientation relative to the user. The registration tool 385 allows for rotational motion of a fiducial contact arm 388, which can be semi-permanently fixed relative to a base plate 92 mounted to the head-wearable device 382 (e.g., a band 85 of the device 382) or directly to the transducer 91. The base plate 92 can then be removed or attached during donning of the head-wearable device 382 to ensure correct position and orientation of the transducer 91 on the head, as described above for the embodiment of the registration tool (i.e., nose fit tool 50) shown by FIGS. 5A and 5B.

As shown in FIG. 12, a rigid fiducial contact arm 388 is mounted on a magnetic base plate 92 through a rotating joint 94. The rotating joint 94 and arm holder 393 can use any mechanical means which allow for free yaw, pitch, or roll, rotation of the fiducial contact arm 388. This may include, but is not limited to, a rotating disc, a ball joint, or a cylinder clamp. During an initial fitting and adjustments procedure, the rotating joint 94 is unlocked allowing for placement of the fiducial contact arm 388 on (e.g., contacting) a given fiducial landmark, as described above for embodiment depicted by FIGS. 5A and 5B. Fiducial landmarks can include corners of the eyes, the nostrils, the tip of the nose, the nose bridge, the lips, the chin, the jawline or any other facial or bodily feature of the user. In the example shown by FIG. 12, the fiducial landmark for the contact arm 388 is a nostril of the user's nose.

Once the arm 388 is correctly in place at the fiducial landmark, the rotating joint 94 can be locked or fixed in place so that it no longer rotates and thus no longer allows the fiducial contact arm 388 to rotate relative to the baseplate 92. The entire registration tool 385, including the fiducial arm 388, the arm holder 393, the rotating joint 94, and the base plate 92 can then be added to the head-wearable device 382 using the magnetic forces from the magnetic material of the base plate 92 to attach the base plate 92 to a metallic surface on the transducer 91 or other portion of the head-wearable device 382 if the registration tool 385 is to be attached at a location other than the transducer 91. The registration tool 385 may be removed from the head-wearable device 382 by pulling the registration tool 385 with sufficient force to overcome the magnetic attraction forces.

Using multiple registration tool structures in this manner, multiple registration points can be created which greatly reduces rotational freedom. As an example, FIG. 12 shows a second registration tool structure having a fiducial contact arm 396 that may be attached to another transducer 397 using a configuration and components described above for the fiducial contact arm 388. The structure of a fiducial contact arm 388, 396 (e.g. shape, curvature, thickness, length, etc.) can be customized for each user or intended fiducial landmark and may have a common connection geometry with the rotating joint 94. In the case of multiple registration tools on a single device, each independent base plate 92 may have a puzzle like fit which only matches its own pieces, barring the user's ability to incorrectly swap the base plate attachment points.

In practice, the user may first attach the registration tool or tools prior to donning the head-wearable device 382. The head-wearable device 382 may then be adjusted until the fiducial arms 388, 396 are contacting the correct fiducial landmarks. The registration tools can then be removed from the device 382 (e.g., detached from the transducers 91, 397) with some force. The user may also leave the device in during treatment to ensure continued accuracy of the wearable position and rotation on the head if they so choose.

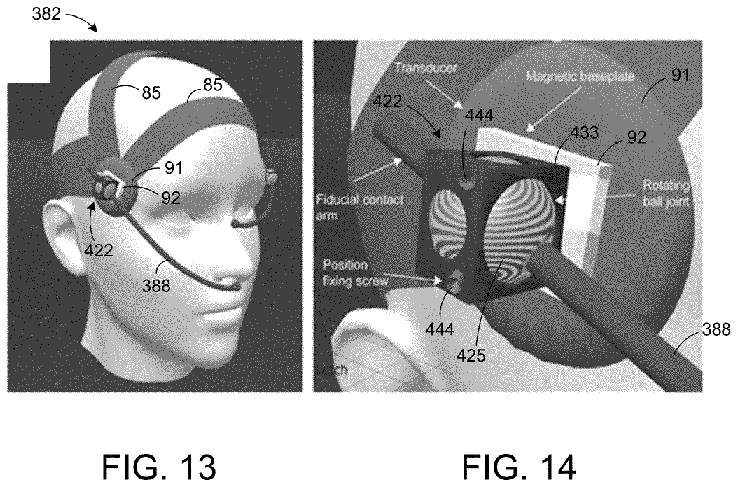

In some embodiments, a rotating ball joint may be used to implement the rotating joint 34 described above. For example, FIG. 13 shows the embodiment of FIG. 12 having a registration tool 421 with a rotating ball joint 422 used in place for the rotating joint 94 and arm holder 393. As shown by FIG. 14, the rotating ball joint 422 has a ball bearing 425 that sits within a cage structure 433 with ample space to allow the ball bearing 425 to rotate freely within the structure 433. The ball bearing 425 contains a cylindrical hole that passes through the center of the structure. The fiducial contact arm 388 passes through this hole. The rotational freedom of the ball bearing 425 (allowing 360° of movement) and the sliding of the fiducial contact arm 388 through the ball bearing 425 allow for highly dynamic fiducial placement on the user's face or body. Custom arms 388 can also have different lengths or shapes. Upon device placement registration, the ball joint 422 and arm 388 can be locked in place using position fixing screws 444 or other types of locking devices. That is, the screws 444 can be screwed into the structure 433 such that they press against the ball bearing 425 with sufficient force to prevent further rotation of the ball bearing 425, thereby locking the ball bearing 425 in place. That is, the ball bearing 425 is locked such that movement of the ball bearing 425 relative to the cage structure 433 and, thus, the base plate 92 and transducer 91 is prevented. Other locking devices may include but are not limited to adhesives, pins, solvents, or clamping devices. After aiding in placement of the head-wearable device 382, the user may remove the tool 421 using magnetic forces, as described above.

The accurate position of the ultrasound transducers with respect to the patient's head is determined from the segmented MRI fiducials and used for the correction of amplitude distortions and phase aberrations introduced by the skull bone in the propagating ultrasound waves. However, after the first MRI scan is concluded, there is no knowledge of the actual transducer position, and if not promptly detected, possible displacements caused by repeated headband wearing can introduce sub-optimal focusing and hamper operational safety. An exemplary method for transducer displacement estimation that enables adaptive aberration correction and improves focusing accuracy is described in detail below.

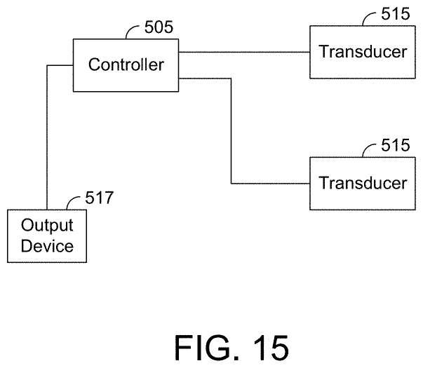

As shown by FIG. 15, a controller 505 may be used to control and receive data from an array of transducers 515, such as transducers that are mounted on a head-wearable device, as described herein. Note that the controller 505 may be implemented in hardware or any combination of hardware and software. As an example, the controller 505 may comprise at least one processor executing software for performing functions ascribed to the controller 505 herein. In other embodiments, the controller 505 may be implemented via a field programmable gate array (FPGA) or application-specific integrated circuit (ASIC). Yet other configurations of the controller 505 are possible in other embodiments.

Following the MRI scanning session when the ultrasound transducers 515 are locked in optimal position, an imaging sequence is executed where short ultrasound pulses (e.g., less than 10 sinusoidal cycles) are emitted by single ultrasound transducers 515 in the array or by combinations thereof. After each pulse is transmitted, the controller 505 switches the transducers 515 to receive mode, and the backscattered ultrasound signals are recorded by combinations of transducers 515 operating in unison. These signals are amplified, sampled, and digitized by the transducers 515, and the digital data are transmitted to the controller 505, which stores the digital data in memory. Following acquisition of an entire ensemble of signals, the data are beam formed by the controller 505 to create a 3-dimensional map of anatomical structures in the region adjacent to the array location. The speed of sound estimated from the MRI data can be used for the calculation of the time-of-flight from each emitting transducer 515 to the point of interest, and back to the receiving transducer 515. The so created anatomical map is then stored and used as reference (“reference map”) for future fine-tuning of the array position with respect to the patient's anatomy.

Later, before a stimulation sequence is run, a new 3-dimensional anatomical map is created like the one described above. Owing to the highly heterogeneous nature of the skull bone, which includes unique local features like notches and diverse thickness, the new map and the reference map can be correlated by the controller 505, for example by calculating cross-correlations, to determine possible transducer displacements. These include rotational and translational displacements that may impair focusing accuracy and reduce operational safety of the ultrasound device. The displacement may be signaled to the user through a visual or auditory cue. In this regard, the controller 505 may be coupled to an output device 517, such as a speaker, light source (e.g., one or more LEDs), or display, that provides audible or visible information indicating whether or an extent to which displacement is detected. Alternatively, the information may be used by the controller 505 to determine the new position and to update the ultrasound or target coordinates in an acoustic simulation. The simulations updated outputs can then correctly steer the beam to the intended target.

It should be noted that the various embodiments described above can be combined as may be desired. As an example, the systems 211 and 133 shown by FIG. 7 or 9 may be used with any of the embodiments of head-wearable devices described herein.

1. A head-wearable device, comprising:
an ultrasound transducer for generating at least one ultrasound pulse for stimulating a brain of a user wearing the head-wearable device;
a registration tool for positioning the ultrasound transducer to a predefined position and orientation relative to the user, the registration tool comprising:
a base plate mounted on the ultrasound transducer; and
a fiducial contact arm coupled to the base plate, the fiducial contact arm extending to and contacting a bodily feature of the user.

2. The head-wearable device of claim 1, wherein the bodily feature is a facial feature of the user.

3. The head-wearable device of claim 1, wherein the bodily feature is a nose of the user.

4. The head-wearable device of claim 1, wherein the registration tool further comprises a rotating joint mounted on the base plate, wherein the fiducial contact arm is held by an arm holder mounted on the rotating joint.

5. The head-wearable device of claim 1, wherein the base plate is composed of magnetic material.

6. The head-wearable device of claim 1, wherein the ultrasound transducer has a recess for receiving the base plate.

7. The head-wearable device of claim 6, wherein a perimeter of the base plate is asymmetrical.

8. The head-wearable device of claim 1, wherein the registration tool further comprises a rotating ball joint mounted on the base plate and coupled to the fiducial contact arm.

9. The head-wearable device of claim 8, wherein the rotating ball joint comprises a ball bearing, and wherein the fiducial contact arm passes through the ball bearing.

10. The head-wearable device of claim 9, further comprising a locking device for locking the ball bearing such that movement of the ball bearing relative to the base plate is prevented.

11. The head-wearable device of claim 1, wherein an end of the fiducial contact arm is coupled to a pad for contacting the bodily feature.

12. The head-wearable device of claim 11, wherein the pad is curved.

13. The head-wearable device of claim 12, wherein the bodily feature is a bridge of a nose of the user.

14. The head-wearable device of claim 1, wherein the fiducial contact arm is forked forming at least a first end for contacting a first fiducial landmark of the user and a second end for contacting a second fiducial landmark of the user.

15. The head-wearable device of claim 1, further comprising:
a first electrode coupled to the ultrasound transducer;
a second electrode coupled to skin of the user; and
a controller configured to measure a parameter indicative of a resistance or capacitance between the first electrode and the second electrode, wherein the controller is configured to provide feedback based on the measured parameter.

16. A method for positioning an ultrasound transducer of a head-wearable device, comprising:
positioning the head-wearable device on a head of a user, the head-wearable device having the ultrasound transducer for generating at least one ultrasound pulse for stimulating a brain of the user; and
positioning the ultrasound transducer to a predefined position and orientation relative to the user via a registration tool having a base plate mounted on the ultrasound transducer and a fiducial contact arm coupled to the base plate, the positioning comprising moving the fiducial contact arm such that the fiducial contact arm contacts a bodily feature of the user.

17. The method of claim 16, wherein the bodily feature is a facial feature of the user.

18. The method of claim 16, wherein the bodily feature is a nose of the user.

19. The method of claim 16, wherein the registration tool further comprises a rotating joint mounted on the base plate, wherein the fiducial contact arm is held by an arm holder mounted on the rotating joint.

20. The method of claim 16, wherein the base plate is composed of magnetic material.

21. The method of claim 16, wherein the ultrasound transducer has a recess for receiving the base plate.

22. The method of claim 21, wherein a perimeter of the base plate is asymmetrical.

23. The method of claim 16, wherein the registration tool further comprises a rotating ball joint mounted on the base plate and coupled to the fiducial contact arm.

24. The method of claim 23, wherein the rotating ball joint comprises a ball bearing, and wherein the fiducial contact arm passes through the ball bearing.

25. The method of claim 24, further comprising locking the ball bearing such that movement of the ball bearing relative to the base plate is prevented.

26. The method of claim 16, wherein an end of the fiducial contact arm is coupled to a pad, and wherein the positioning the ultrasound transducer to the predefined position or orientation comprises contacting the bodily feature with the pad.

27. The method of claim 26, wherein the pad is curved.

28. The method of claim 27, wherein the bodily feature is a bridge of a nose of the user.

29. The method of claim 16, wherein the fiducial contact arm is forked forming at least a first end and a second end, and wherein the positioning the ultrasound transducer to the predefined position and orientation comprises:
contacting a first fiducial landmark of the user with the first end; and
contacting a second fiducial landmark of the user with the second end.

30. The method of claim 16, further comprising:
measuring a parameter indicative of a resistance or capacitance between a first electrode coupled to the ultrasound transducer and a second electrode coupled to skin of the user; and
providing feedback based on the measured parameter.
